(19) United States
(12) Patent Application Publication
(10) Pub. No.: US 2003/0157205 A1
(43) Pub. Date: Aug. 21, 2003

(54) Inhibitory and preventative effects of processed morinda citrifolia on mutagenesis and carcinogenesis in mammals

(76) Inventors: Claude Jarakae Jensen, Cedar Hills, UT (US); Chen Su, West Jordan, UT (US); Mian-Ying Wang, Rockford, IL (US); Summer Jensen, Cedar Hills, UT (US); Johnathan William Fritz, Orem, UT (US); Stephen P. Story, Alpine, UT (US)

Correspondence Address: KIRTON & McCONKIE, 1800 Eagle Gate Tower, 60 East South Temple, Salt Lake City, UT, 84111, US

(21) Appl. No.: 10/335,653

(22) Filed: Dec. 31, 2002

Related U.S. Application Data
(60) Provisional application No. 60/344,707, filed on Dec. 31, 2001.

(51) Int. Cl. A61K 35/78

(52) U.S. Cl. 424/765; 424/769; 424/777

(57) ABSTRACT

The present invention features a method for inhibiting, reducing, and/or preventing mutagenesis, or inducing antimutagenesis activity, through the prophylactic administration of a naturaceutical formulation comprising at least one of a processed Morinda citrifolia product. The present invention seeks to provide a natural composition, or naturaceutical, for the purpose of inhibiting both naturally occurring or spontaneous mutagenesis, as well as environmentally induced mutagenesis, within the body of a mammal.

### RELATED APPLICATIONS

This application claims priority to U.S. Provisional Application Serial No. 60/344,707, filed Dec. 31, 2001, and entitled, “Methods of Using Processed Morinda citrifolia as an Anti-oxidant and to Inhibit Mutagenesis Within the Cells of Mammals.”

### BACKGROUND

1. Field of the Invention

The present invention relates to methods and formulations for inhibiting and preventing mutagenesis or rather inducing antimutagenesis and anticarcinogenesis activity in mammals, as well as repairing existing DNA damage.

2. Background of the Invention and Related Art

Mutagenesis occurs within DNA and cell development either spontaneously, or naturally, or it may be induced as a side-effect of chemicals, high-energy radiation, stress, etc. Mutagens are agents that cause mutations. Under experimental conditions they are also carcinogens (i.e. capable of inducing cancer) and teratogens (capable of causing birth defects). Mutagens induce DNA damage that either kills cells or, when unrepaired, produces abnormal sequences that will be passed on to daughter cells. These actions, in turn, induce birth defects by injuring developing organs or by initiating mosaicism that disorganizes growth and differentiation.

Society is now more aware and concerned about the presence of mutagens in the environment. Mutagens are ubiquitous; some are naturally present in plants, many are produced by combustion of organic materials (including cooking), and others are products of industry.

People today face many health problems. Many of these health problems result from cell and DNA damage within the body caused by mutagens and mutagenesis. Factors contributing to cell and DNA damage include, among others, pollution, stress, aging, cigarette smoke, ultraviolet light, excessive exercise, and joint and tissue injuries. Because of this cell damage, many diseases and illnesses wreak havoc on the human body. Some of these diseases and illnesses resulting from cell damage include: age spots, ageing, allergies, Alzheimer's, angina, arthritis, asthma, atherosclerosis, bleeding gums, internal bleeding, bruise, cancer, cataracts, circulation problems, cirrhosis, cold feet, cold fingers, diabetes type II, dementia, dry skin, fatigue, hay fever, hardening of the arteries, heart attacks, hemorrhoids, hypertension, inflamed tissues, jet lag, kidney damage, liver damage, liver disease, male sexual inadequacy, memory loss, menstrual disorders, migraine headaches, multiple sclerosis, night blindness, Parkinson's disease, phlebitis, prostate problems, psoriasis, respiratory problems, retinopathy, rheumatism, senility, skin cancers, strokes, stress damage, swollen joints, swollen limbs, varicose veins, and wrinkling of the skin.

While pharmaceutical compositions exist to induce antimutagenesis activity within the body, these are not without sometimes significant side effects.

As such, what is needed is an improved natural food product and method of using the same for the purpose of inhibiting and preventing mutagenesis and mutagen activity within the body, as well as repairing existing DNA and cellular damage.

### SUMMARY AND OBJECTS OF THE INVENTION

In light of the foregoing, the present invention seeks to provide a natural composition, or naturaceutical, for the purpose of inhibiting both naturally occurring or spontaneous mutagenesis, as well as environmentally induced mutagenesis, within the body of a mammal. Stated differently, the primary focus of the present invention is to provide a naturaceutical composition formulated for the specific purpose of providing or inducing antimutagenesis effects within the body of a mammal. The present invention also functions to provide anticarcinogenesis effects.

Antimutagenesis may be referred to as the cumulative processes or by which cells reduce the frequency of mutations. These processes could be important in reducing the frequency of bacterial mutations to drug/antibiotic resistance in decreasing the frequency of the mutations that lead to some types of cancers, and even in addressing the mutations that lead to genetic illnesses and may play a role in aging.

In accordance with the invention as embodied and broadly described herein, the present invention features a method for inhibiting, reducing, and/or preventing mutagenesis, or inducing antimutagenesis activity, through the prophylactic administration of a naturaceutical formulation comprising at least one of a processed Morinda citrifolia product. The Processed Morinda citrifolia product may be in the form of fruit juice, puree juice, fruit or puree juice concentrate, oil extract, dietary fiber, or one of an alcohol or aqueous extract.

The naturaceutical formulation may be embodied in any form suitable for systemic internalization, namely orally, topically or transdermally, or intravenously. Moreover, the naturaceutical formulation may contain various amounts and concentrations of a processed Morinda citrifolia product, along with other various ingredients, such as a carrier medium or composition in a topical dermal naturaceutical formulation.

In accordance with the invention as embodied and broadly described herein, the present invention features a naturaceutical formulation or composition that comprises as an active ingredient, one or more processed Morinda citrifolia products, which function to inhibit, reduce, and/or prevent mutagenesis by blocking or inhibiting DNA adduct formation.

To practice the invention, the Morinda citrifolia-based naturaceutical formulation or composition is administered to a candidate or patient exhibiting one or more of the signs of cell damage in an amount sufficient to eliminate or at least inhibit or alleviate one or more of the signs or symptoms. The preferred dosage is at least two ounces of processed Morinda citrifolia (preferably in fruit juice or puree juice form) administered at least twice daily for a sustained period of time, preferably at least two months. Morinda citrifolia may also be administered in non-juice form, such as in a solid or powder form (e.g., using a dietary fiber product).

The present invention further features a method for administering the processed Morinda citrifolia-based naturaceutical formulation concurrently with a pharmaceutical designed to inhibit and prevent mutagenesis and carcinogenesis. The naturaceutical formulation functions to increase the efficacy of such pharmaceuticals.

Using the methods and dosages described herein and in the claims, the function of the present invention processed Morinda citrifolia is to alleviate the symptoms and inhibit the proliferation of many diseases and illness, including, age spots, aging, allergies, Alzheimer's, angina, arthritis, asthma, atherosclerosis, bleeding gums, internal bleeding, bruise, cancer, cataracts, circulation problems, cirrhosis, cold feet, cold fingers, diabetes type II, dementia, dry skin, fatigue, hay fever, hardening of the arteries, heart attacks, hemorrhoids, hypertension, inflamed tissues, jet lag, kidney damage, liver damage, liver disease, male sexual inadequacy, memory loss, menstrual disorders, migraine headaches, multiple sclerosis, night blindness, Parkinson's disease, phlebitis, prostate problems, psoriasis, respiratory problems, retinopathy, rheumatism, senility, skin cancers, strokes, stress damage, swollen joints, swollen limbs, varicose veins, and wrinkling of the skin.

Processed Morinda citrifolia also works to protect cells in many ways. For example, processed Morinda citrifolia can protect white blood cells from radiation damage, liver cells from lipid peroxidation and vascular endothelial cells from oxidant injury. Processed Morinda citrifolia also enhances antioxidative enzyme systems in cells and inhibits the formation of TBA-RS. Processed Morinda citrifolia inhibits multiple proatherogenic and prothrombotic oxidative events in the artery wall. Through its interaction with cells and DNA, processed Morinda citrifolia reduces risk of coronary disease and reduces restenosis after angioplasty.

Through these functions, it is set forth that processed Morinda citrifolia has a very positive effect on diseases and illnesses that result from mutated DNA. Pollution causes a reaction inside human bodies that injures DNA. By neutralizing pollutants, processed Morinda citrifolia stops the chain reaction of DNA injury. Taking processed Morinda citrifolia will lower DNA mutagen sensitivity. As people age, DNA damage becomes more frequent. Toxins generated by infection also cause DNA damage. A DNA lesion that is not properly repaired can give rise to a mutation. Such DNA damage contributes to the increased incidence of cancers and other chronic diseases. It has been discovered that introducing processed Morinda citrifolia into the body functions to significantly repair existing DNA damage, as well as provide a deterrent against further DNA damage.

Moreover, an important factor in mutagenesis, and therefore carcinogenesis, is the cell division rate in the precursors of tumor cells. Increasing the cell division rate increases mutations. However, administering processed Morinda citrifolia to a patient can decrease mutagenesis, and therefore carcinogenesis, in two ways—namely, by decreasing oxidative DNA damage and by decreasing the overall division rates of tumor cells.

### DETAILED DESCRIPTION OF THE PREFERRED EMBODIMENTS

It will be readily understood that the components of the present invention, as generally described and illustrated in the figures herein, could be arranged and designed in a wide variety of different configurations. Thus, the following more detailed description of the various embodiments of formulations and methods of the present invention is not intended to limit the scope of the invention, as claimed, but is merely representative of the presently preferred embodiments of the invention.

The presently preferred embodiments of the invention will be best understood by separating the description into sections, the first pertaining to a general discussion regarding Morinda citrifolia and the methods employed to produce and manufacture the processed Morinda citrifolia products used as key ingredients in the formulations described herein; and the second being a more detailed and specific discussion on the methods and formulations used to treat DNA mutations and inhibit and prevent further mutagenesis using one or more of the processed Morinda citrifolia-based naturaceutical products as described herein, including examples of experimental studies and the results obtained.

### General Discussion of Morinda citrifolia and the Methods used to Produce Processed Morinda citrifolia Products

The Indian Mulberry or Noni plant, known scientifically as Morinda citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been used by several nationalities as a food source, in past times, the most common use of the Morinda citrifolia plant was as a red and yellow dye. Only recently has there been an interest in the nutritional and health benefits of the Morinda citrifolia plant, which are further discussed below.

Because the Morinda citrifolia fruit is, for all practical purposes, inedible the fruit must be processed in order to make it palatable for consumption by a mammal, and especially consumption by humans. The fruit is also processed to optimize its ability to inhibit and prevent mutagenesis within mammals and to be better suited to be included in food products used for the same.

Processed Morinda citrifolia fruit juice can be prepared by separating seeds and peels from the juice and pulp of a ripened Morinda citrifolia fruit, filtering the pulp from the juice, and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

In one exemplary embodiment, the present invention comprises a naturaceutical composition that utilizes the fruit juice and/or puree juice extracted from the Morinda citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product, either in dilute or concentrate form. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.) and may exist in concentrated or diluted form.

As stated, another product manufactured is processed Morinda citrifolia puree and puree juice, in either concentrate or diluted form. Puree is essentially the pulp a separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

In another exemplary embodiment, the processed Morinda citrifolia product may also exist as a dietary fiber, which may be incorporated into a naturaceutical composition suitable for the purpose of inhibiting, reducing, and preventing mutagenesis.

In another exemplary embodiment, the processed Morinda citrifolia product may comprise an oil or exist in oil form, which may be incorporated into a suitable naturaceutical composition that utilizes the processed Morinda citrifolia oil to inhibit, reduce, and/or prevent mutagenesis. The method for extracting and processing the oil is described in co-pending application Ser. No. 09/384,785, filed on Aug. 27, 1999, which is hereby incorporated by reference herein. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used. It should be noted that many of the naturaceutical compositions described and claimed herein may comprise combinations of Morinda citrifolia products, namely fruit juice, puree juice, dietary fiber, and/or oil, in various amounts.

The Morinda citrifolia plant is rich in natural ingredients. Those ingredients that have been discovered include: from the leaves: alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; from the flowers: acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; from the fruit: acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; from the roots: anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; from the root bark: alizarin, chlororubin, glycosides (pentose, hexose), morindadiol, morindanigrine, morindine, morindone, resinous matter, rubiadin monomethyl ether, and soranjidiol; from the wood: anthragallol-2,3-dimethylether; from the tissue culture: damnacanthal, lucidin, lucidin-3-primeveroside, and morindone-6beta-primeveroside; from the plant: alizarin, alizarin-alpha-methyl ether, anthraquinones, asperuloside, hexanoic acid, morindadiol, morindone, morindogenin, octanoic acid, and ursolic acid.

Recently, as mentioned, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

As a result of its many recognized health benefits, Morinda citrifolia has been used to provide a number of anecdotal effects in individuals and to treat various ailments ranging from cancer, arthritis, headaches, indigestion, malignancies, broken bones, high blood pressure, diabetes, pain, infection, asthma, toothaches, blemishes, immune system failure, and others.

The present invention naturaceutical compositions containing one or more processed Morinda citrifolia products may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets preferably contain Morinda citrifolia in admixture with non-toxic pharmaceutically acceptable excipients, which are suitable for the manufacture of tablets. These excipients may be, for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions preferably contain the processed Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, the present invention provides a method of inhibiting, reducing, and preventing mutagenesis or DNA mutation within mammals through the prophylactic administration of a naturaceutical formulation comprising one or more processed Morinda citrifolia products without any significant tendency to cause gastric or other undesirable side effects.

### Preventative Effects of Morinda citrifolia on Mutagenesis and DNA Mutagens

The present invention generally describes a formulation and method for inhibiting and preventing mutagenesis, and therefore carcinogenesis, within a mammal. The present invention particularly describes a formulation comprising Morinda citrifolia in processed form and a method of using the same for repairing DNA damage caused by pollutants, toxins, infections, and other mutagen activating and inducing factors, preventing further DNA damage, reducing DNA mutagen sensitivity, repairing DNA legions, decreasing oxidative DNA damage, and decreasing the overall division rates of various tumor cells. Each of these is described below in greater detail. Other related effects that are not specifically recited herein will be recognized and apparent to one ordinarily skilled in the art.

The present invention advances mutagenesis inhibitors, and therefore carcinogenesis inhibitors and methods (otherwise known as antimutagenesis and anticarcinogenesis products and methods), by providing a naturaceutical treatment or composition formulated with one or more processed Morinda citrifolia products as derived from the Indian Mulberry plant. The processed Morinda citrifolia product(s) is/are incorporated into various naturaceutical compositions suitable for in vivo treatment of a patient. For instance, the naturaceutical may be ingested, introduced via an intravenous injection or feeding, applied topically and transdermally internalized, or otherwise internalized into the body as is appropriate and directed.

The present invention describes the function of the Morinda citrifolia-based naturaceutical formulations in their described dosages and methods of administration to alleviate the symptoms and inhibit the proliferation of many diseases and illness caused by DNA mutations, including age spots, ageing, allergies, Alzheimer's, angina, arthritis, asthma, atherosclerosis, bleeding gums, internal bleeding, bruise, cancer, cataracts, circulation problems, cirrhosis, cold feet, cold fingers, diabetes type II, dementia, dry skin, fatigue, hay fever, hardening of the arteries, heart attacks, hemorrhoids, hypertension, inflamed tissues, jet lag, kidney damage, liver damage, liver disease, male sexual inadequacy, memory loss, menstrual disorders, migraine headaches, multiple sclerosis, night blindness, Parkinson's disease, phlebitis, prostate problems, psoriasis, respiratory problems, retinopathy, rheumatism, senility, skin cancers, strokes, stress damage, swollen joints, swollen limbs, varicose veins, and wrinkling of the skin.

The present invention further describes the function of the Morinda citrifolia-based naturaceutical formulations and methods of administration to protect the cells of mammals in various ways. For instance, processed Morinda citrifolia protects white blood cells from radiation damage, liver cells from lipid peroxidation, and vascular endothelial cells from oxidant injury; enhances antioxidative enzyme systems in cells and inhibits the formation of Thiobarbituric Acid Reactive Substances (TBA-RS); and inhibits multiple proatherogenic and prothrombotic oxidative events in the artery wall. Moreover, through its interaction with cells and DNA, processed Morinda citrifolia reduces risk of coronary disease and reduces restenosis after angioplasty.

Still further, the present invention describes the positive effect of the Morinda citrifoliabased naturaceutical formulations and methods of administration on diseases and illnesses that result from mutated DNA.

Activation of mutagenesis, and subsequent DNA mutations, within the body occurs as a result of either natural or unnatural causes. Natural mutations occur as a result of improper DNA replication during the reproduction process and are not the primary focus of the present invention. On the other hand, unnatural activation or induced mutagenesis and subsequent DNA mutations within the body occurs as a result of many factors, the primary causes being exposure to various toxins, pollutants, and other harmful substances found in environmental conditions, overly strenuous and prolonged physical exertion.

Environmentally induced mutagenesis is frequently the result of exposure to various pollutants, toxins, etc. that are introduced into the body and function as mutagens to destroy DNA and disrupt basic cellular functions. As such, introduction of unsafe amounts of pollutants and toxins into the body can cause harmful reactions that injure DNA strands and cause mutations within those strands, thus contributing to the onset of disease. For example, upon metabolic activation, environmental polycyclic aromatic carcinogens can react with DNA to form carcinogen-DNA adducts. These adducts may initiate cancer by inducing mutagenesis, thus causing mutations during DNA replication. This type of environmentally induced mutagenesis is commonly referred to as carcinogenesis, which results in the generation and proliferation of various carcinogenic or cancerous cells within the body.

Administration of the present invention naturaceutical formulation functions to neutralize these pollutants, therefore stopping the chain reaction of DNA injury. Introducing processed Morinda citrifolia further into the body functions to lower DNA mutagen sensitivity, thus decreasing the effect that these harmful mutagens can have on DNA. The less these mutagens are able to effect the DNA, the less likely mutagenesis or carcinogenesis will be activated, which in turn decreases the likelihood of becoming diseased.

Another factor contributing to mutagenesis is age. As mammals age, DNA damage becomes more frequent. Throughout a mammals lifespan the body will be infected with several toxin generating microorganisms. These toxins invade DNA strands and cause damage in the form of lesions. A DNA lesion that is not properly repaired can subsequently give rise to a mutation. Such DNA damage contributes to the increased incidence of cancers and other chronic diseases. However, through administration of the present invention naturaceutical formulation using the methods described herein, DNA damage, namely DNA lesions, can be repaired. Moreover, the Morinda citrifolia present in the naturaceutical functions to reduce the effects of harmful toxins by increasing the resistance capabilities of the DNA structure, as well as to function as a deterrent to prevent the onset of additional DNA damage by causing the DNA to become less sensitive to foreign substances, such as toxins and pollutants.

It is known in the art that an important factor in mutagenesis, and therefore carcinogenesis, is the cell division rate in the precursors of tumor cells. Increasing the cell division rate increases mutations. However, administering processed Morinda citrifolia to a patient can decrease mutagenesis, and therefore carcinogenesis, in two ways—namely, by decreasing oxidative DNA damage and by decreasing the overall division rates of tumor cells. This is explained in greater detail in Example Two below.

In one exemplary embodiment, the naturaceutical composition of the present invention comprises one or more processed Morinda citrifolia products present in an amount by weight between about 0.011 and 100 percent, and preferably between about 0.01 and 95 percent by weight. Several embodiment of formulations are provided below. However, these are only intended to be exemplary, as one ordinarily skilled in the art will recognize other formulations or compositions capable of comprising the processed Morinda citrifolia product(s).

The processed Morinda citrifolia product is designed to function as the active ingredient or at least to contain one or more active ingredients, such as Quercetin and Rutin, and others, for the purpose of effectuating the inhibition and prevention of mutagenesis within the body, for effectuating the repair of damaged DNA strands, as well as for inhibiting the generation and proliferation of cancerous or otherwise diseased or infected cells. If desired, the various active ingredients of Quercetin and Rutin may be extracted out using alcohol or alcohol-based solutions, such as methanol, ethanol, and ethyl acetate, and other alcohol-based derivatives using any known process in the art. Quercetin and Rutin are preferably present in amounts by weight ranging from 0.01-10 percent of the total formulation or composition. However, these amounts may be concentrated as well into a more potent concentration in which they are present in amounts ranging from 10 to 100 percent.

As indicated, the processed Morinda citrifolia product may be formulated with various other ingredients, natural or artificial, to produce various compositions or formulations, in addition to a strictly all-natural naturaceutical composition. As such, the term naturaceutical is intended to encompass any formulation that comprises at least an amount of processed Morinda citrifolia as an active ingredient, as well as a significant portion of natural ingredients. Any additional or supplemental ingredients to be utilized or incorporated into the naturaceutical or composition are any that are safe for introduction into the body of a mammal, and particularly a human. In addition, the naturaceutical composition or formulation may exist in various forms, such as a liquid, tablets, lozenges, an aqueous or oily solution, dispersible powders or granules, emulsions, syrups, elixirs, etc. Moreover, since the Morinda citrifolia-based composition will most likely be consumed orally, it may contain one or more agents selected from the group consisting of sweetening agents, flavoring agents, coloring agents, preserving agents, and other medicinal agents as directed.

The present invention naturaceutical may also be in the form of a topical dermal composition. The ingredients to be utilized in a topical dermal composition are also any that are safe for internalizing into the body of a mammal and may exist in various forms, such as gels, lotions, creams, ointments, etc., each comprising one or more carrier agents. The ingredients for systemically administered formulations may also comprise any that are known in the art.

In one exemplary embodiment, the present invention features a method of administering a naturaceutical composition to a mammal for the inhibition and prevention of mutagenesis. The method comprises the steps of (a) formulating a naturaceutical composition comprising in part a processed Morinda citrifolia product present in an amount between about 0.01 and 95 percent by weight, wherein the composition also comprises a carrier, such as water or purified water, and other natural or artificial ingredients; (b) introducing the naturaceutical composition into the body such that the processed Morinda citrifolia product is sufficiently internalized and concentrated therein; (c) repeating the above steps as often as necessary to provide an effective amount of the processed Morinda citrifolia product to inhibit and prevent mutagenesis, or induce antimutagenesis effects, within the body.

In one preferred, yet exemplary embodiment, the above step of administering the naturaceutical composition into the body comprises ingesting the composition orally through one of several means. Specifically, the naturaceutical composition may be formulated as a liquid, gel, solid, or some other type that would allow the composition to be quickly digested and internalized. It is important to note that the step of administering the naturaceutical composition should be carried out in an effective manner so that the greatest concentration of naturaceutical composition is allowed to be internalized. For the naturaceutical composition to take effect, it must be sufficiently internalized, thus allowing the processed Morinda citrifolia product or active ingredient to properly effectuate any antimutagenesis activity. Once sufficiently internalized, it may then begin to function and perform as indicated and described herein.

In an alternative, yet exemplary embodiment, the step of administering the naturaceutical composition may include injecting the composition into the body using an intravenous pump. This technique is advantageous as it would allow the composition to be localized in the area where it would have the most effect, or within the area that would most benefit from the presence of the concentration of the naturaceutical composition.

In one exemplary embodiment, the method of administering the naturaceutical composition comprises taking between 1 teaspoon and 2 oz., and preferably 2 oz., of the naturaceutical composition every two hours each day, or at least twice a day. Also, the naturaceutical composition is to be taken on an empty stomach, meaning at a period of time at least two hours prior to consumption of any food or drink. Following this, the naturaceutical composition is allowed to actively effectuate antimutagenesis activity within the body, thereby inhibiting and preventing DNA mutations, as well as repairing existing any DNA damage that might cause a mutation. Of course, one ordinarily skilled in the art will recognize that the amount of composition and frequency of use may vary from individual to individual. However, the present invention contemplates always administering a safe, pre-determined dosage of the naturaceutical for a safe, pre-determined duration of time, such as in the aforementioned exemplary embodiment.

The following tables illustrate or represent some of the preferred formulations or compositions contemplated by the present invention. As stated,:these are only intended as exemplary embodiments and are not to be construed as limiting in any way.

| | |
|---|---|
| Ingredients | Percent by weight |

| | |
|---|---|
| Formulation One | |
| Morinda citrifolia puree juice or fruit juice | 100% |
| Formulation Two | |
| Morinda citrifolia fruit juice | 85-99.99% |
| Water | 0.1-15% |
| Formulation Three | |
| Morinda citrfolia fruit juice | 85-99.99% |
| non-Morinda citrifolia-based fruit juices | 0.1-15% |
| Formulation Four | |
| Morinda citrifolia fruit juice | 50-90% |
| Water | 0.1-50% |
| non-Morinda citrifolia-based fruit juices | 0.1-30% |
| Formulation Five | |
| Morinda citrifolia puree juice | 85-99.9% |
| Water | 0.1-15% |
| Formulation Six | |
| Morinda citrifolia puree juice | 85-99.9% |
| non-Morinda citrifolia-based fruit juices | 0.1-15% |
| Formulation Seven | |
| Morinda citrifolia puree juice | 50-90% |
| Water | 0.1-50% |
| non-Morinda citrifolia-based fruit juices | 0.1-30% |
| Formulation Eight | |
| Morinda citrifolia dietary fiber | 0.1-30 % |
| Water | 1-99.9% |
| non-Morinda citrifolia-based fruit juices | 1-99.9% |
| Formulation Nine | |
| Morinda citrifolia dietary fiber | 0.1-30% |
| Water | 1-99.9% |
| Morinda citrifolia fruit juice or puree juice | 1-99.9% |
| Formulation Ten | |
| Morinda citrifolia oil | 0.1-30% |
| carrier medium | 70-99.9% |
| other ingredients | 1-95% |
| Formulation Eleven | |
| Morinda citrifolia product | 10-80% |
| carrier medium | 20-90% |
| Formulation Twelve | |
| Morinda citrifolia product | 5-80% |
| carrier medium | 20-95% |
| Formulation Thirteen | |
| Morinda citrifolia oil or oil extract | 0.1-20% |
| carrier medium | 20-90% |
| Formulation Fourteen | |
| Morinda citrifolia puree juice or fruit Juice | 0.1-80% |
| Morinda citrifolia oil | 0.1-20% |
| carrier medium | 20-90% |
| Formulation Fifteen | |
| Morinda citrifolia puree juice concentrate or | 100% |
| fruit juice concentrate | |
| Formulation Sixteen | |
| Morinda citrifolia fruit juice concentrate or | 85-99.99% |
| puree juice concentrate | |
| Water | 0.1-15% |
| Formulation Seventeen | |
| Morinda citrifolia fruit juice or puree juice | 0.01-99.99% |
| Methyl sulfonyl methane | 0.01-99.99% |

In one preferred method, a patient desiring to inhibit and prevent mutagenesis and/or to repair existing DNA damage, as well as treat other related conditions, takes, or is administered, at least one (1) ounce of one of Formulations One through Seven in the morning on an empty stomach, and at least one (1) ounce at night on an empty stomach, just prior to retiring to bed. In one example, such as with Formulation One, which is not meant to be limiting in any way, the beneficial Morinda citrifolia is processed into a naturaceutical composition known as Tahitian Noni® juice manufactured by Morinda, Incorporated of Orem, Utah.

As stated, in one exemplary embodiment, the present invention features a method for introducing, internally into the body, a naturaceutical composition or formulation to inhibit and prevent mutagenesis and DNA damage. This method essentially comprises the introduction, in any way, of the naturaceutical composition into cells having existing DNA damage to function as a repair mechanism, and into other cells without damage to function as a deterrent. Several embodiments of the naturaceutical composition comprising various different ingredients are contemplated for use herein, with each embodiment comprising at least one form of a processed Morinda citrifolia product as taught and explained herein.

In one exemplary embodiment, the ingredients of the naturaceutical composition may be comprised of a processed Morinda citrifolia product present in an amount by weight between about 10-80 percent; and a carrier medium present in an amount by weight between about 20-90 percent, wherein the carrier medium is preferably water or other fruit juices. In this embodiment, the processed Morinda citrifolia product may comprise one or more of processed Morinda citrifolia fruit juice (in dilute or concentrate form), processed Morinda citrifolia puree juice (in dilute or concentrate form), processed Morinda citrifolia dietary fiber, and/or processed Morinda citrifolia oil extract.

In another exemplary embodiment, the ingredients of the naturaceutical composition may be comprised of processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. The Morinda citrifolia puree juice or fruit juice may also be formulated with a Morinda citrifolia dietary fiber product in similar concentrations.

The carrier medium described above may comprise any ingredient capable of being introduced into the body of a mammal, and that is also capable of providing the means or the carrying medium for the processed Morinda citrifolia product. Different carrier mediums are contemplated, such as one providing for a topical dermal preparation (e.g. a lotion or cream), or one embodying the Morinda citrifolia dietary fiber, etc. Specific carrier mediums formulations are well known in the art and not described in detail herein. The purpose of the carrier medium is as stated, to provide a means to embody the processed Morinda citrifolia product within the naturaceutical composition, wherein the carrier medium itself is capable of being introduced and internalized into the body.

According to the present invention, these particular methods of introducing the naturaceutical composition into the body may comprise any method. For instance, some of the preferred methods of introducing the naturaceutical into the body are intravenously, transdermally, orally, or systemically. However, no matter what method is employed, it is important that the naturaceutical is properly internalized so that the active ingredients existing in the naturaceutical can effectively function as intended.

In addition, although the time period for administering such dosages should be for a period of at least two months, greater or less time periods may be warranted. Moreover, greater dosages may be administered, as there is no threat of undesirable, harmful, or adverse side effects stemming from the Morinda citrifolia product(s). Indeed, increased dosages have been found to be significantly more beneficial and are preferred. For example, in one experiment, up to one liter of Tahitian Noni® fruit juice was administered daily. During the treatment period, the fruit juice induced within the patient a significant prophylactic effect with no adverse side effects. In addition, anecdotal evidence indicates that some remedial benefits may also be experienced.

The following examples set forth and present the effects of Morinda citrifolia on both pre-existing headaches, as well as the preventative effects of Morinda citrifolia against the onset of future headaches. These examples are not intended to be limiting in any way, but are merely illustrative of the beneficial, advantageous, and remedial effects of Morinda citrifolia on headaches, and particularly migraine headaches. Other non-limiting examples of the present invention are described below.

### EXAMPLE ONE

This study indicates the preventative effects of Methyl Sulfonyl Methane (MSM), processed Morinda citrifolia products (e.g., Tahitian Noni juice), and their combination on estrogen-induced mammary carcinogenesis in female ACI rats.

In the study, the preventative effects of MSM, Morinda citrifolia, and their combination on mammary gland carcinogenesis induced by 17 β-estradiol (estrogen)(E2) pellet implants in female ACI rats was studied. In the experiment, sixty (60), five week old female rats were divided into four groups, fifteen (15) rats each, and placed on regular diets. Another eight (8) female ACI rats were served as the age-matched controls. Five percent placebo, five percent Morinda citrifolia, five percent MSM, a combination of five percent Morinda citrifolia and MSM in drinking water was supplied to the animals in different groups until ninety days after E2 implantation. Two weeks later, all animals were implanted subcutaneously with a 25 mg pellet containing 22.5 mg of 17 β-estradiol (e2) mixed with 2.5 mg cholesterol, and the age-matched control animals received a 25 mg cholesterol pellet implant.

The animals in the placebo group had a significant body weight loss when compared to the cholesterol control group. The animals in the Morinda citrifolia or MSM group had slight body weight loss. None of the rats that received pellets composed of cholesterol exhibited mammary tumors. All rats with an E2 implant in the placebo group had mammary glad tumors. One hundred percent of the rats in this group had three to seven tumors. Seventy-one percent of the rats in the Morinda citrifolia group had two to five tumors, fifty-seven percent of the rats in the MSM group had one to four tumors, and forty-three percent of the rats in the combination group had zero to three tumors. The average tumor area in the placebo group, 5% MSM, 5% Morinda citrifolia, and 5% MSM and Morinda citrifolia combination groups at 180 days after E2 implantation was 17, 12, 10, and 6 mm2, respectively. The survival rates of the control, 5% placebo, 5% Morinda citrifolia, and 5% MSM and Morinda citrifolia combination groups at 160 days after E2 implantation was 100%, 0%, 47%, 73%, and 87%, respectively. The survival rate of these different groups at 180 days was 100%, 0%, 0%, 20%, and 60%, respectively. At 200 days, the survival rates of these different groups was 100%, 0%, 0%, 0%, and 27%, respectively. This data indicates that MSM was able to prevent mammary glad tumor at the initiation stage of carcinogenesis induced by E2 and its synergetic effect with 5% Morinda citrifolia.

In light of the foregoing, a naturaceutical formulation comprising Morinda citrifolia and MSM in any combination is contemplated herein. Moreover, a method of inhibiting and preventing mutagenesis in mammals is also contemplated herein, wherein the method comprises the steps of administering a naturaceutical formulation comprising one or more processed Morinda citrifolia products present in an amount by weight between about 0.01 and 100 percent and MSM present in an amount by weight between about 0.01 and 100 percent. Of course, other ingredients and food products may be added to the formulation with their respective percentages by weight present as discussed herein.

### EXAMPLE TWO

Oxidants in human body attack lipids, sugars, proteins, and DNA and induce their oxidation, which may result in oxidative damage such as membrane dysfunction, protein modification, enzyme inactivation, and break of DNA strands and modification of its bases. Antioxidant status is the balance between the antioxidant system and pro-oxidants in human body system. The body is slightly imbalance favoring oxidation. A serious imbalance favoring oxidation is defined as oxidative stress and can develop into sever diseases including heart disease and cancer.

During evaluation of Morinda citrifolia antioxidant activities, a lipid hydroperoxide assay was used to test Morinda citrifolia for the activity of scavenging lipid hydroperoxides. The assay principle was a combination of cumen hydroperoxide and leucomethylene blue to produce methylene blue (absorb UV light at 660 nm).

The Morinda citrifolia assay (tetrazolium nitroblue) (TNB) was used to test for the activity of scavenging superoxide anion radicals. The principle used was a combination of superoxide anion radical and TNB to form formzan blue (absorb UV light at 602 nm). As there are two types of oxidants in the human body, namely peroxides and free radicals, Morinda citrifolia assays were tested to analyze their antioxidant activities towards these two types of oxidants.

The studies concluded that Morinda citrifolia is an antioxidant to both lipid hydroperoxide and superoxide anion radical. It was also found that Morinda citrifolia antioxidant activities were comparable to Vitamin C (Morinda citrifolia being 2.8 stronger), pycnogenols (Morinda citrifolia is 1.4 times stronger), and grape seed powder (Morinda citrifolia is 1.1 times stronger) at their suggested daily intake amount. It was also discovered that the antioxidant activities of Morinda citrifolia did not diminish over a four year period of time.

This study illustrates the significant antioxidant activities associated with the processed Morinda citrifolia product described herein to contribute to the inhibition and prevention of mutagenesis.

The present invention further features a method of inhibiting and preventing mutagenesis and repairing existing DNA damage comprising taking, or administering to a patient, a naturaceutical formulation comprising one or more processed Morinda citrifolia products as described herein, concurrently with one or more antimutagenesis pharmaceuticals or medications. Taking the naturaceutical composition concurrently with an antimutagenesis medication functions to increase the efficacy of the antimutagenesis medication, thus giving rise to greater, more effective treatment of mutagenesis activity. The ability of the naturaceutical formulation, and particularly the Morinda citrifolia product contained therein, to increase the efficacy of the migraine medication results from the cellular enhancement capabilities of the processed Morinda citrifolia, thus allowing a greater amount of the active ingredients in the antimutagenesis medication is allowed to be absorbed into the cells. In addition, the Morinda citrifolia product is very active against or inhibit the drug breakdown functions of cytochrome enzymes, which are responsible for the breaking down and metabolizing of drugs. As a result, drugs are allowed to be in the body much longer, which naturally allows them to be more effective. Taking or administering the naturaceutical formulation comprising one form or another of a processed Morinda citrifolia product as taught and described herein not only functions to enhance the relief potential for the patient by increasing or enhancing the efficacy of the antimutagenesis medication, but it also provides the same benefits and advantages to the patient that are obtained directly from the naturaceutical formulation itself.

The present invention may be embodied in other specific forms without departing from its spirit of essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method for inhibiting and preventing mutagenesis in mammals, said method comprising the steps of:
introducing a naturaceutical composition into the body of said mammal, said naturaceutical composition comprising a processed Morinda citrifolia product present in an amount between about 0.01 and 100 percent by weight, said processed Morinda citrifolia product comprising the active ingredient Quercetin; and
administering a safe, pre-determined amount of said naturaceutical composition for a safe, pre-determined duration of time.

2. The method of claim 1, wherein said Quercetin is present in an amount between about 0.1 and 10 percent by weight.

3. The method of claim 1, wherein said processed Morinda citrifolia product is Morinda citrifolia fruit juice.

4. The method of claim 1, wherein said processed Morinda citrifolia product is Morinda citrifolia oil extract.

5. The method of claim 1, wherein said processed Morinda citrifolia product is Morinda citrifolia dietary fiber.

6. The method of claim 1, wherein said processed Morinda citrifolia product is Morinda citrifolia puree juice.

7. The method of claim 1, wherein said processed Morinda citrifolia product is Morinda citrifolia puree.

8. The method of claim 1, wherein said processed Morinda citrifolia product further comprises Rutin as an additional active ingredient that synergistically works with said Quercetin to inhibit and prevent mutagenesis.

9. The method of claim 8, wherein said Rutin is present in an amount between about 0.1 and 10 percent by weight.

10. The method of claim 1, wherein said composition is administered orally.

11. The method of claim 1, wherein said composition is administered transdermally to said infected area.

12. The method of claim 1, wherein said composition is administered by injection.

13. The method of claim 1, wherein said composition is administered intravenously.

14. The method of claim 1, wherein said composition is administered systemically.

15. The method of claim 1, wherein said processed Morinda citrifolia product comprises processed Morinda citrifolia fruit juice concentrate.

16. The method of claim 1, wherein said processed Morinda citrifolia product comprises processed Morinda citrifolia puree juice concentrate.

17. The method of claim 1, wherein said naturaceutical composition functions to block DNA adduct formation.

18. A method for inducing antimutagenesis activity within mammals and repairing existing DNA damage, said method comprising the steps of:
adding a processed Morinda citrifolia product to an alcohol-based solution;
isolating and extracting an active ingredient of said processed Morinda citrifolia product from said solution;
introducing, internally, said extracted active ingredient into the body of said mammal, said active ingredient of said Morinda citrifolia functions to inhibit and block DNA adduct formation, thus inducing antimutagenesis activity within said mammal.

19. The method of claim 18, wherein said processed Morinda citrifolia product comprises processed Morinda citrifolia fruit juice.

20. The method of claim 18, wherein said processed Morinda citrifolia product comprises processed Morinda citrifolia puree.

21. The method of claim 18, wherein said alcohol-based solution is selected from the group consisting essentially of methanol, ethanol, and ethyl acetate, and other alcohol-based derivatives.

22. The method of claim 18, wherein said active ingredient is Quercetin.

23. The method of claim 22, wherein said active ingredient is Rutin that synergistically works with said Quercetin to inhibit and prevent growth of said cancerous cells in said colon region.

24. The method of claim 18, wherein said step of introducing, internally, said extracted active ingredient into said body of said mammal comprises orally administering two ounces of a food product comprising said active ingredient to said mammal twice daily each day on an empty stomach.

25. The method of claim 18, wherein said step of introducing, internally, said extracted active ingredient into said body of said mammal comprises applying a topical composition comprising said active ingredient to the epidermis of said mammal.

26. A method for inducing antimutagenesis activity within mammals, said method comprising the steps of:
orally administering at least one ounce of a naturaceutical formulation comprising a processed Morinda citrifolia product on an empty stomach in the morning; and
orally administering to said mammal at least one ounce of said naturaceutical formulation on an empty stomach prior to sleeping at night.

27. The method of claim 26, wherein said naturaceutical formulation comprises:
processed Morinda citrifolia fruit juice present in an amount by weight of about 100 percent.

28. The method of claim 26, wherein said naturaceutical formulation comprises:
processed Morinda citrifolia fruit juice present in an amount by weight between about 85-99.99 percent; and
water present in an amount by weight between about 0.1-15 percent.

29. The method of claim 26, wherein said naturaceutical formulation comprises:
processed Morinda citrifolia fruit juice present in an amount by weight between about 85-99.99 percent; and
non-Morinda citrifolia-based fruit juices present in an amount by weight between about 0.1-15 percent.

30. The method of claim 26, wherein said naturaceutical formulation comprises:
processed Morinda citrifolia fruit juice present in an amount by weight between about 50-90 percent;
water present in an amount by weight between about 0.1-50 percent; and
non-Morinda citrifolia-based fruit juices present in an amount between about 0.1-30 percent.

31. The method of claim 26, wherein said naturaceutical formulation comprises:
processed Morinda citrifolia puree juice present in an amount by weight of about 100 percent.

32. The method of claim 26, wherein said naturaceutical formulation comprises:
processed Morinda citrifolia puree juice present in an amount by weight between about 85-99.99 percent; and
water present in an amount by weight between about 0.1-15 percent.

33. The method of claim 26, wherein said naturaceutical formulation comprises:
processed Morinda citrifolia puree juice present in an amount by weight between about 85-99.99 percent; and
non-Morinda citrifolia-based fruit juices present in an amount by weight between about 0.1-15 percent.

34. The method of claim 26, wherein said naturaceutical formulation comprises:
processed Morinda citrifolia puree juice present in an amount by weight between about 50-90 percent;
water present in an amount by weight between about 0.1-50 percent; and
non-Morinda citrifolia-based fruit juices present in an amount between about 0.1-30 percent.

35. The method of claim 26, wherein said step of orally administering comprises orally administering two ounces of said naturaceutical formulation.

36. A method for inhibiting and preventing mutagenesis in mammals, said method comprising the steps of:
orally administering at least one ounce of a naturaceutical formulation comprising a processed Morinda citrifolia product in the morning on an empty stomach;
orally administering at least one ounce of said naturaceutical formulation prior to sleeping at night;
repeating said steps of orally administering said naturaceutical for a sustained safe, pre-determined duration of time to effectuate antimutagenesis activity within said mammal.

37. The method of claim 36, wherein said naturaceutical formulation comprises:
processed Morinda citrifolia fruit juice present in an amount between about 100 percent by weight.

38. The method of claim 36, wherein said naturaceutical formulation comprises:
processed Morinda citrifolia fruit juice present in an amount between about 85-99.99 percent by weight; and
water present in an amount between about 0.1-15 percent by weight.

39. The method of claim 36, wherein said naturaceutical formulation comprises:
processed Morinda citrifolia fruit juice present in an amount between about 85-99.99 percent by weight; and
non-Morinda citrifolia-based fruit juices present in an amount between about 0.1-15 percent by weight.

40. The method of claim 36, wherein said naturaceutical formulation comprises:
processed Morinda citrifolia fruit juice present in an amount between about 50-90 percent by weight;
water present in an amount between about 0.1-50 percent by weight; and
non-Morinda citrifolia-based fruit juices present in an amount between about 0.1-30 percent by weight.

41. The method of claim 36, wherein said naturaceutical formulation comprises:
processed Morinda citrifolia puree juice present in an amount of about 100 percent by weight.

42. The method of claim 36, wherein said naturaceutical formulation comprises:
processed Morinda citrifolia puree juice present in an amount between about 85-99.99 percent by weight; and
water present in an amount between about 0.1-15 percent by weight.

43. The method of claim 36, wherein said naturaceutical formulation comprises:
processed Morinda citrifolia puree juice present in an amount between about 85-99.99 percent by weight; and
non-Morinda citrifolia-based fruit juices present in an amount between about 0.1-15 percent by weight.

44. The method of claim 36, wherein said naturaceutical formulation comprises:
processed Morinda citrifolia puree juice present in an amount between about 50-90 percent by weight;
water present in an amount between about 0.1-50 percent by weight; and
non-Morinda citrifolia-based fruit juices present in an amount between about 0.1-30 percent by weight.

45. The method of claim 36, wherein said step of orally administering comprises orally administering two ounces of said naturaceutical formulation twice daily each day for a safe, pre-determined duration of time.

46. A method for inhibiting and preventing mutagenesis in mammals, said method comprising the steps of:
administering a naturaceutical composition to a mammal, said naturaceutical composition comprising:
at least one processed Morinda citrifolia product present in an amount between about 0.01 and 100 percent by weight; and
Methyl sulfonyl methane present in an amount between about 0.01 and 100 percent by weight.

47. A method for decreasing oxidative DNA damage, said method comprising the steps of:
administering a naturaceutical composition to a mammal, said naturaceutical composition comprising:
at least one processed Morinda citrifolia product present in an amount between about 0.01 and 100 percent by weight.

48. A method for decreasing the division rate of tumor cells, said method comprising the steps of:
administering a naturaceutical composition to a mammal, said naturaceutical composition comprising:
at least one processed Morinda citrifolia product present in an amount between about 0.01 and 100 percent by weight.

49. A method for inhibiting and preventing mutagenesis within a mammal, said method comprising the steps of:
introducing an internal composition into the body of said mammal, said internal composition comprising:
at least one processed Morinda citrifolia product present in an amount between about 10-80 percent by weight; and
a carrier medium present in an amount between about 20-90 percent by weight.

50. The method of claim 49, wherein said processed Morinda citrifolia product is selected from the group consisting of processed Morinda citrifolia fruit juice, processed Morinda citrifolia puree juice, processed Morinda citrifolia dietary fiber, and processed Morinda citrifolia oil and oil extract.

51. The method of claim 49, wherein said step of introducing an internal composition comprises a method selected from the group consisting of intravenously, injection, transdermally, orally, and systemically.

52. A method for inhibiting and preventing mutagenesis and repairing existing DNA damage within a mammal, said method comprising the steps of:
introducing an internal composition into the body of said mammal, said internal composition comprising:
at least one processed Morinda citrifolia product present in an amount by weight between about 5-80 percent; and
a carrier medium present in an amount by weight between about 20-95 percent.

53. The method of claim 53, wherein said processed Morinda citrifolia product is selected from the group consisting of processed Morinda citrifolia fruit juice, processed Morinda citrifolia puree juice, processed Morinda citrifolia dietary fiber, processed Morinda citrifolia oil, and processed Morinda citrifolia oil extract.

54. The method of claim 53, wherein said step of introducing an internal composition comprises a method selected from the group consisting of intravenously, injection, transdermally, orally, and systemically.

55. A method for inhibiting and preventing mutagenesis and repairing existing DNA damage within a mammal, said method comprising the steps of:
introducing an internal composition into the body of said mammal, said internal composition comprising the ingredients of:
processed Morinda citrifolia oil present in an amount by weight between about 0.1-20 percent; and
a carrier medium present in an amount by weight between about 80-99.9 percent.

56. A method for inhibiting and preventing mutagenesis and repairing existing DNA damage within a mammal, said method comprising the steps of:
introducing an internal composition into the body of said mammal, said internal composition comprising the ingredients of:
processed Morinda citrifolia product selected from one of a fruit juice product and a puree juice produce, said processed Morinda citrifolia product present in an amount by weight between about 0.1-80 percent;
processed Morinda citrifolia oil present in an amount by weight between about 0.1-20 percent; and
a carrier medium present in an amount by weight between about 20-90 percent.

57. A method for inhibiting the drug breakdown functions of cytochrome enzymes for the purpose of facilitating antimutagenesis activity within a mammal, said method comprising the steps of:
administering an antimutagenesis medication to a patient;
administering a naturaceutical composition to said patient concurrently with said antimutagenesis medication, said naturaceutical comprising a processed Morinda citrifolia product that increases the efficacy of said antimutagenesis medication by inhibiting the medication breakdown and metabolizing functions of said cytochrome enzymes, said processed Morinda citrifolia product present in an amount by weight between 0.1 and 100 percent.

58. A naturaceutical composition for inhibiting and preventing mutagenesis and repairing DNA damage in mammals, said naturaceutical composition comprising:
at least one processed Morinda citrifolia product present in an amount between about 0.01 and 100 percent by weight.

59. The composition of claim 58, wherein said processed Morinda citrifolia product comprises Morinda citrifolia fruit juice.

60. The composition of claim 58, wherein said processed Morinda citrifolia product comprises Morinda citrifolia puree juice.

61. The composition of claim 58, wherein said processed Morinda citrifolia product comprises Morinda citrifolia puree juice concentrate.

62. The composition of claim 58, wherein said processed Morinda citrifolia product comprises Morinda citrifolia fruit juice concentrate.

63. The composition of claim 58, wherein said processed Morinda citrifolia product comprises Morinda citrifolia dietary fiber.

64. The composition of claim 58, wherein said processed Morinda citrifolia product of said naturaceutical composition further comprises an active ingredient Quercetin present in an amount between about 0.1 and 10 percent by weight.

65. The composition of claim 64, wherein said processed Morinda citrifolia product further comprises Rutin as an additional active ingredient that synergistically works with said Quercetin to induce antimutagenesis activity within said mammal.

66. The composition of claim 65, wherein said Rutin is present in an amount between about 0.1 and 10 percent by weight.

67. The composition of claim 58, wherein said naturaceutical composition is administered via a method selected from the group consisting of oral administration, transdermal administration, intravenous administration, systemic administration, and administration by injection.

68. A naturaceutical formulation for effectuating antimutagenesis activity within mammals, said naturaceutical formulation comprising:
at least one processed Morinda citrifolia product present in an amount by weight between about 0.01 and 100 percent; and
Methyl Sulfonyl methane present in an amount by weight between about 0.01 and 100 percent.
